(19) United States
(12) Patent Application Publication
(10) Pub. No.: US 2021/0153858 A1
(43) Pub. Date: May 27, 2021

(54) SURGICAL RETRACTOR FOR ROBOTIC AND/OR LAPAROSCOPIC SYSTEMS

(71) Applicant: Sarah King, San Francisco, CA (US)

(72) Inventor: Sarah King, San Francisco, CA (US)

(21) Appl. No.: 17/095,034

(22) Filed: Nov. 11, 2020

Related U.S. Application Data
(60) Provisional application No. 62/941,141, filed on Nov. 27, 2019.

(51) Int. Cl. A61B 17/02; A61B 34/30

(52) U.S. Cl. CPC A61B 17/0206; A61B 2017/0225; A61B 34/30; A61B 17/0218

(57) ABSTRACT

A retractor of a robotic and/or laparoscopic surgery system is provided. The retractor may support in a generally uniformly distributed manner weight and/or massing of retracted internal patient tissue. The retractor typically comprises an arm extendable from the system terminating in a pair of graspers, and a weight distributing means located between the pair of graspers for distributing weight of retracted tissue, so as to avoid injury to the retracted tissue during deployment of the retractor. The graspers may have open and closed positions. The weight distributing means may comprise, e.g., a netting or a fabric, having a tautness effective to effect grasping and/or retraction action during deployment of the retractor. For example, the weight distributing means may in the alternative comprise a fan mechanism that optionally includes the pair of graspers. One or more surgical procedures may be performed using that retractor in a robotic and/or laparoscopic technique.

### CROSS REFERENCE TO RELATED CASES

This application claims priority to U.S. Provisional Patent Application No. 62/941,141, filed Nov. 29, 2019, entitled “SURGICAL RETRACTOR FOR ROBOTIC AND/OR LAPAROSCOPIC SYSTEMS” the disclosure of which is incorporated by reference in its entirety.

### BACKGROUND

There are a number of robotic surgery systems in the marketplace. An example of a robotic surgery system is the Da Vinci system manufactured by Intuitive Surgical, Inc. (Sunnyvale, Calif.)

Robotic surgeries involve surgical procedures that are performed using robotic systems. Robotically-assisted surgery was developed to try to overcome the limitations of pre-existing minimally-invasive procedures and to enhance the capabilities of surgeons performing open surgery.

In the case of robotically-assisted minimally-invasive surgery, instead of directly moving the instruments, a surgeon uses one of a number of methods to administer the instruments. These include using a direct telemanipulator or through computer control.

A telemanipulator is a remote manipulator that allows the surgeon to perform the normal movements associated with the surgery. Robotic arms carry out those movements using end-effectors and manipulators to perform the actual surgery. Telemanipulators can also be an extension of a robotic arm.

In computer-controlled systems, the surgeon uses a computer to control the robotic arms and its end-effectors, though these systems can also still use telemanipulators for their input. One advantage of using the computerized method is that the surgeon does not have to be physically present, leading to the possibility for remote surgery.

In this patent application, laparoscopy refers to a surgical procedure in which a fiber-optic instrument is inserted through small incisions into the abdomen, pelvis, chest, or other internal space to view the internal anatomy or to permit a surgical procedure. When compared with traditional surgery, laparoscopic surgical procedures usually have: less pain following the procedure; lower risk of complications; a shorter hospital stay and a quicker recovery; and a much smaller scar.

A retractor is a surgical instrument used to separate the edges of a surgical incision or wound, or to hold back underlying organs and tissues so that body parts under the incision may be accessed. Retractors are often required for robotic surgical procedures as well as open surgery.

Retractors are not unknown in the art. For example, the following papers generally describe retractors known in the art. See, e.g., Alkatout (2018), “An atraumatic retractor for interdisciplinary use in conventional laparoscopy and robotic surgery,” MINIMALLY INVASIVE THERAPY & ALLIED TECHNOLOGIES 27:5, pp. 265-271; and Kobayashi et al. (2016), “Utility of an Internal Retractor (EndoGrab) for the Management of the Vesicouterine Ligament during Laparoscopic Radical Hysterectomy,” GYNECOL OBSTET INVEST 8 pp.363-366.

One problem associated with current retractors, e.g., for robotic and/or laparoscopic systems, is that they are not designed for weakened and/or friable tissue. As a result, unnecessary injury may occur during robot surgery procedures.

Thus, opportunities exist to provide improvements in the art for retractors for use in robotic and laparoscopic surgery systems.

### SUMMARY

A retractor of a robotic and/or laparoscopic surgery system is provided. When a robotic system is provided, the system optionally comprises a surgeon console, a patient cart, and a vision cart. The retractor may exhibit a multipurpose nature and may be effective to support in a generally uniformly distributed manner weight and/or massing of retracted tissue located within an animal, e.g., a human, or a nonhuman animal in need of surgery. The retractor comprises an arm extendable from the system terminating in a pair of outer members, e.g., graspers, and a weight distributing means located between the pair of outer members for distributing weight of retracted tissue, so as to avoid injury to the retracted tissue during deployment of the retractor.

The outer members may have open, closed and adjustable positions. The positions may be achieved by having graspers pivoting about an axis substantially perpendicular to a direction of arm extension relative to the system.

The weight distributing means may be of variable structures, for instance may comprise a netting or a fabric having a tautness effective to effect grasping and/or retraction action during deployment of the retractor. Or, for example, the weight distributing means may comprise a fan mechanism that optionally includes the pair of outer members. The pair of outer members may represent outer slats of the fan mechanism during deployment of the retractor. For example, the weight distribution means may comprise a fan mechanism having a plurality of overlapping slats.

The retractor of the invention may be detachable from the robotic surgery system. When attached to the robotic surgery system, the retractor may be effective to carry out a laparoscopic surgical procedure. Alternatively, the retractor of the invention may be used in laparoscopic but non-robotic surgical applications.

The graspers may have a width of about 1 cm to about 30 cm and a length of about 3 cm to about 15 cm. The graspers, in some embodiments of the invention may have teeth. Alternatively, the retractor may be provided absent sharp edges. The size of the inventive instruments may vary depending on the surgery to be performed and the incision to be made before the instrument is deployed.

In another embodiment, a retractor is provided that may be used with a robotic and/or laparoscopic surgery system. The retractor has a distal end and a proximal end. When a robotic surgery system is provided, the proximal end is located closer to a portion of the system located closer to a surgeon console. The retractor is effective to support in a generally uniformly distributed manner weight and/or massing of retracted tissue located within an animal. The retractor comprises outer members, e.g., a first grasper and a second grasper located at the distal end of the retractor. The graspers may achieve open and closed positions, and a netting or a fabric is located between the first and second graspers. The netting or fabric have a tautness effective to effect grasping and/or retraction action during deployment of the retractor.

Further provided is a retractor as generally described above. The retractor has a distal end and a proximal end, the proximal end located closer to a portion of the system located closer to a surgeon console. The retractor is effective to support in a generally uniformly distributed manner weight and/or massing of retracted tissue located within an animal. The retractor includes an arm extendable from the system terminating in a fan mechanism having a plurality of overlapping slats for distributing weight of retracted tissue, so as to avoid injury to the retracted tissue during deployment of the retractor.

One or more surgical procedures may be performed using that retractor of the invention. For example, the surgical procedures may involve a laparoscopic technique. In addition or in the alternative, the procedures include but are not limited to any surgery requiring retraction, such as cardiac, colorectal, general, gynecological, thoracic, and urologic surgery.

### BRIEF DESCRIPTION OF THE DRAWINGS

FIG. 1 depicts in an open position a retractor of the invention having a fan-type construction.

FIG. 2 depicts in a closed position a retractor of the invention shown in FIG. 1.

FIG. 3 depicts in an open position a retractor of the invention having a net-type construction.

FIG. 4 depicts in a closed position a retractor of the invention shown in FIG. 3.

FIG. 5 depicts a component of the inventive retractor having hooks to which netting or fabric may be attached.

### DETAILED DESCRIPTION OF THE INVENTION

Before describing the present invention in detail, it is to be understood that the invention is not limited to specific robotic and/or laparoscopic surgical systems or robotic modules, as such may vary. It is also to be understood that the terminology used herein is for the purpose of describing particular embodiments only, and is not intended to be limiting.

In addition, as used in this specification and the appended claims, the singular article forms “a,” “an,” and “the” include both singular and plural referents unless the context clearly dictates otherwise. Thus, for example, reference to “a slat” includes a single slat as well as a plurality of slats, reference to “a robot” refers to a single robot as well as a plurality of robots, and the like.

In this specification and in the claims that follow, reference will be made to a number of terms that shall be defined to have the following meanings, unless the context in which they are employed clearly indicates otherwise:

The term “grasper” is used in its ordinary sense and refers to an item that in combination with other parts of the invention may be used to hold or otherwise seize tissue firmly in a retracting manner.

The term “netting” is used herein to refer to meshed, open or closed, material made by interlaying or interweaving together wire, monofilament, thread or the like. Nettings may also be formed from unwoven plastic films and/or biologics. Other forms of netting will be apparent from the perspective of persons of ordinary skill in the art.

The term “retractor” is used in its ordinary surgical sense and refers to a surgical instrument used to separate the edges of a surgical incision or wound, or to hold back underlying organs and tissues so that body parts under the incision may be accessed.

The term “slat” is use herein to refer to parts of the inventive retractor that typically comprise a thin, narrow pieces of material, such as plastic, metal, or ceramics, but not limited to these materials, especially one of a series which overlap or fit into each other.

Thus, the invention generally relates to retractors for use with robotic systems and/or laparoscopic procedures, as generally described above and set forth in the claims below. As a general matter, surgical retractors include a member that during deployment includes a curved, hooked, or angled section that maintains the desired position of a given region of tissue. The invention involves one or more retractors intended for use at the end of a robotic arm. Optionally, retractors may be fitted both with suction and with fiber optic lights to keep a surgical wound dry and illuminated.

The invention may have a closed position before deployment. In such a case, the retractor may have a generally elongated cylindrical shape. Once deployed, a distal end of the retractor may expand to distribute the weight and/or massing of retracted tissue. In such a case, graspers open to result in an expanded netting or fan-like manner so as to avoid injury to the retracted tissue during deployment of the retractor.

Deployment and withdrawal of the inventive retractor may involve mechanical, electrical, thermal, fluidic, etc., or a combination of such actions. As a result, angular, rotational, lengthening, shortening, expansion, shrinkage, and/or combinations of movements may occur as appropriate during deployment and/or withdrawal of the retractor from the surgical field. Optionally, simple machines such as screws, levers, pulleys, and the like may be used to effect such movements.

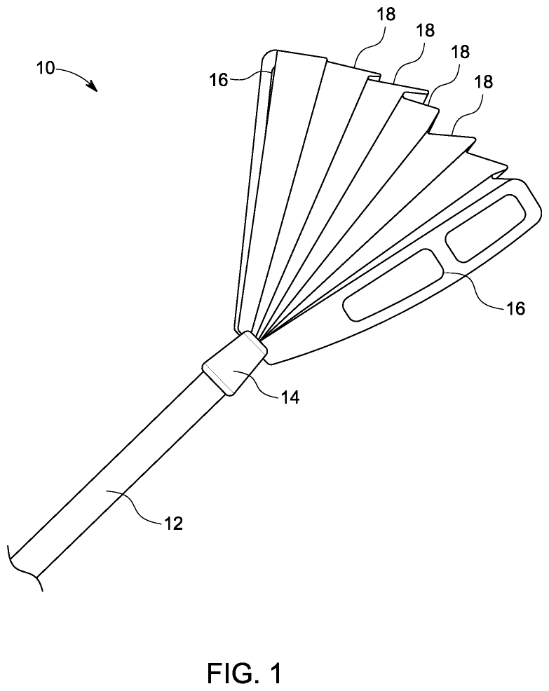

As shown in FIG. 1, an embodiment of the inventive retractor may be deployed and manipulated in situ to exhibit an open position. As with all figures referenced herein, in which like parts are referenced by like numerals, FIG. 1 is not necessarily to scale, and certain dimensions may be exaggerated for clarity of presentation. As shown, the retractor 10 includes an arm 12 terminating at a trocar 14, outer graspers 16, and slats 18 arranged in a fan like positioning between the graspers 16. Typically, the graspers do not have teeth, but teeth e.g., serrations, may be provided to allow the retractor to exhibit greater frictional forces for immobilizing retracted tissue during surgery. Also typically, as shown in FIG. 1, the arm may be straight, but curved or bent arms, e.g., telescoping and other variants of arms may be apparent upon experimentation in conjunction with existing or future robotic and/or laparoscopic systems.

As shown in FIG. 1, the fan-type construction allows slats to open up in an overlapping fan like manner. Typically, the slats 18, when opened, do not exhibit any gaps therebetween to reduce the likelihood of injury to tissue upon use. Avoidance of construction with gaps between slats can reduce the likelihood of snags relative to a patient's internal tissue.

Injury associated with surgical tool use is typically measured by biochemical and/or enzymatic presence in the blood (or possibly urine). Thus, the invention also provides a means for conducting surgery so as to minimize certain biochemical and/or enzymatic presence (e.g., liver enzymes) in a patient's blood stream. Detection of such biochemical and/or enzymatic presence in the blood stream may be performed by phlebotomists and/or medical professionals.

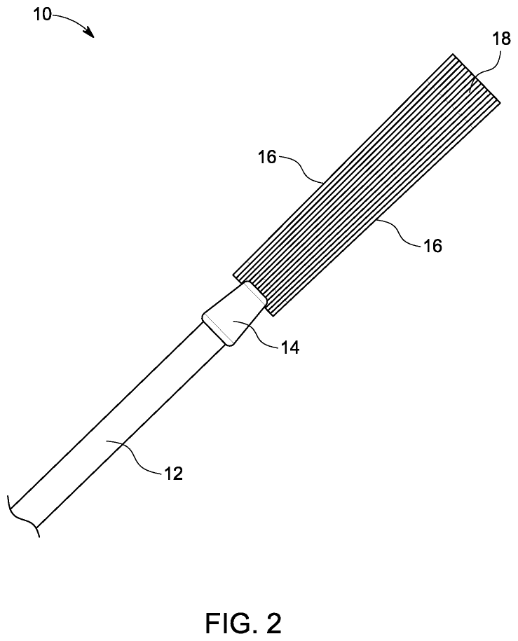

FIG. 2 shows the inventive retractor depicted in FIG. 1. As shown, the retractor is in a closed position. The collapsibility of the retractor allows the retractor in closed positioned to be easily manipulated or moved before and/or after deployment in a surgical field.

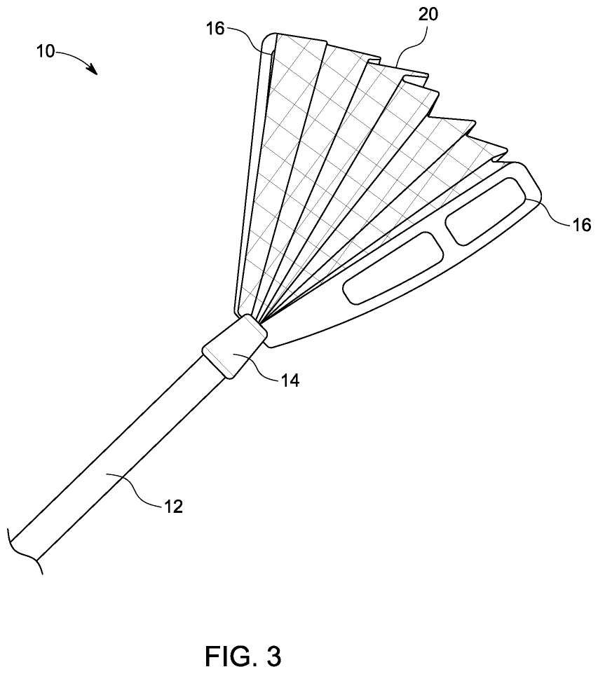

FIG. 3 shows another embodiment of the inventive retractor 10 may also be deployed in an open position. In such a case, the weight distributing means 20 may not have a fan-like construction. In particular, a netting, mesh, fabric or like material 20 may be provided having a tautness between graspers 14 effective to effect grasping and/or retraction action during deployment of the retractor.

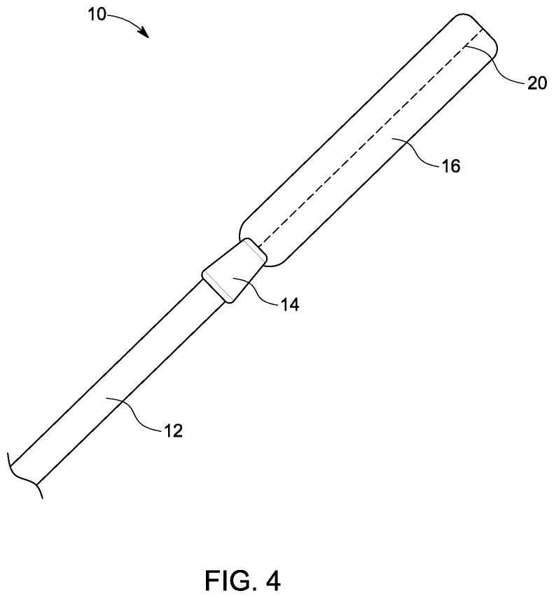

As shown in FIG. 4, once the surgery is completed, the retractor 10 may be rendered in a closed position once more for removal from the surgical field. In some instances, the weight distributing means 20 may be folded within the trocar 14 and the arm 12, when the retractor 10 is in a position. Typically, the retractor of the invention is intended for a single use, thought multiple use retractors are not necessarily excluded from the invention unless explicitly recited in the claims.

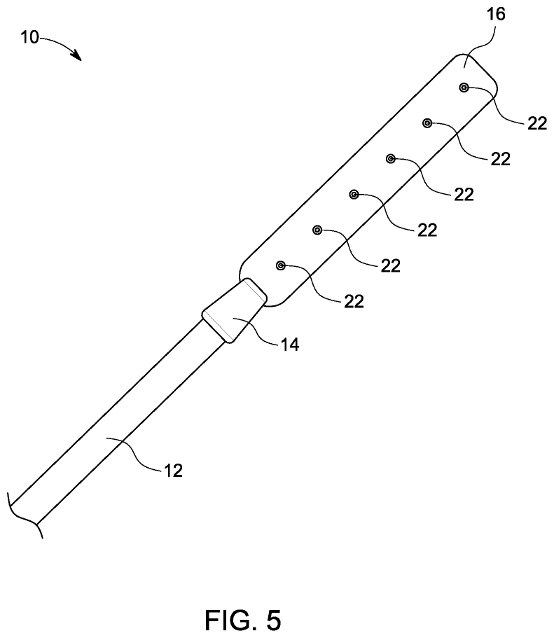

In general, the invention may be designed for single use or multiple use. In some instances, the portions of the invention may be designed and/or constructed for single use while other parts of the invention may be designed and/or constructed for multiple use, e.g., after sterilization before and after each use. As shown in FIG. 5, hooks 22 along the length of a grasper 16 may be used to attach netting and/or fabric to the grasper 16. In such a case, the netting and/or fabric may be considered single use while the graspers 16 may be a part of the invention that are constructed for multiple use.

Means for attachment other than hooks 22 may be employed. Such means may include, for example, loops, adhesives, welds, mating couplings, etc.

Variations of the present invention will be apparent to those of ordinary skill in the art in view of the disclosure contained herein. For example, the invention may contain or exclude specific features according to the intended functionality of the inventive surgical retractor. Additional variations of the invention may be discovered upon routine experimentation without departing from the spirit of the present invention. A light source may be provided to allow the surgeon to visualize the surgical field in laparoscopic and/or robotic surgeries.

Similarly, the invention may employ slats, nettings, and variants thereof such as films and the like to support the weight or massing of tissue retracted. In some embodiments, flexible materials may be used. For example, a flexible central material may be plastic, metal, biofilm or biodegradable material constructed in a woven or solid form as long as it is sturdy enough to maintain retraction while flexible enough to mold to organs for best weight distribution and safety.

In short, the invention provides a retractor to be used in laparoscopic and robotic surgeries. It is a safer and more effective retracting device than what is currently available. In some embodiments, the invention is designed as a fan-like instrument that may be introduced through port sites while collapsed and then expanded inside the body for use. It functions to limit organ and tissue injury while providing better exposure by retracted large surface areas. The device may be used on all internal organs and pathology, including but not limited to the stomach, spleen, liver, gallbladder, small bowel, large bowel, uterus, ovaries, kidneys, bladder, tumors and cysts.

The length of the graspers may be 3-30 cm and the width of the netting may be up to 20 cm all depending on what requires retracting. The graspers spread apart to open the mesh between them. They also have small teeth for functional grasping when necessary. The netting (consistency in definition) between the graspers is without large holes in order to best distribute the weight what is being retracted across the whole device in order to avoid injury from too much localized pressure. It will be flexible enough to mold to the shape of the given organ while stiff enough to provide safe and effective support.

There is an instrument called a liver retractor that is effective at retracting large organs, such as the liver, for better visualization of other intra-abdominal organs. A problem with the liver retractor is that it risks injury to the organs that it retracts. The rigidity of the instrument and the concentrated pressure on the retracted organ leads to damage that can be grossly visualized during a procedure or indicated by lab value changes. Another current retracting technique is to use a bowel grasper to grab and pull tissue out of the way. This technique risks injury when grasping and it is not always effective for large and/or floppy tissue. The laparoscopic fan is another retracting device that risks internal injury. The fans have sharp edges and corners in the device that risk injury. Internal tissue can be fragile, and when there is inflammation or pathology involved, then tissue is especially friable and vulnerable to injury with the devices listed above. The inventive device is envisioned to be more gentle on tissues while also providing more effective retraction and visualization during laparoscopic and robotic surgery.

The invention is a safer and more effective tool for retraction of the liver, bowel, intraabdominal masses and any other tissues that may obstruct exposure in laparoscopic or robotic cases. The fan-like design distributes weight more evenly than a liver retractor, minimizing the risk of injury. The fan-like design also enables large masses or amounts of tissue to be retracted effectively by a single instrument. It will be useful to retract the liver in bariatric surgical cases, replacing the liver retractor. It will be useful to retract bowel or masses in gynecological surgeries. It will be a safe and effect way to retract in any other intraabdominal or intrapelvic procedures that require retraction of organs or masses to provide improved visualization.

The design of the invention can be reproduced to different sizes to be most appropriate for a given case. The fan width and grasper length can be variable. There will be a mesh or netted fan material for flexible retraction of more delicate tissues and there will be a slatted, metal fan that will be more adjustable but rigid.

It is to be understood that, while the invention has been described in conjunction with the preferred specific embodiments thereof, the foregoing description merely illustrates and does not limit the scope of the invention. Numerous alternatives and equivalents exist which do not depart from the invention set forth above. Other aspects, advantages, and modifications within the scope of the invention will be apparent to those skilled in the art to which the invention pertains.

What is claimed is:

1. A retractor effective to support in a generally uniformly distributed manner weight and/or massing of retracted tissue located within an animal, comprising:
an arm extendable from a system terminating in a pair of graspers, and
a weight distributing means located between the pair of graspers for distributing weight of retracted tissue, so as to avoid injury to the retracted tissue during deployment of the retractor.

2. The retractor of claim 1, wherein the graspers have open, closed and adjustable positions that are achieved by the graspers pivoting about an axis substantially perpendicular to a direction of arm extension relative to the system.

3. The retractor of claim 1, wherein the weight distributing means comprises a netting, a film, or a fabric having a tautness effective to effect grasping and/or retraction action during deployment of the retractor.

4. The retractor of claim 1, wherein the weight distributing means comprises a fan mechanism that between the pair of graspers.

5. The retractor of claim 4, wherein the pair of graspers represent outer slats of the fan mechanism during deployment of the retractor.

6. The retractor of claim 1, wherein the weight distribution means comprises a fan mechanism having a plurality of overlapping slats.

7. The retractor of claim 1, provided as a component of a robotic surgery system, and the retractor is detachable from the robotic surgery system.

8. The retractor of claim 1, effective to carry out a laparoscopic surgical procedure.

9. The retractor of claim 1, wherein the graspers have a width of about 1 cm to about 30 cm.

10. The retractor of claim 9, wherein the graspers have a length of about 3 cm to about 15 cm.

11. The retractor of claim 1, wherein the graspers have teeth.

12. The retractor of claim 1, absent sharp edges.

13. A retractor of a robotic and/or laparoscopic surgery system, the retractor having a distal end and a proximal end, the proximal end located closer to a portion of the system located closer to a surgeon console, retractor effective to support in a generally uniformly distributed manner weight and/or massing of retracted tissue located within an animal, comprising:
a first grasper and a second grasper located at the distal end of the retractor, wherein the graspers may achieve open, closed and adjustable positions, and
a netting or a fabric located between the first and second graspers, wherein the netting or fabric have a variable tautness effective to effect grasping and/or retraction action during deployment of the retractor.

14. The retractor of claim 13, wherein the graspers have teeth.

15. The retractor of claim 13, absent sharp edges.

16. A retractor of a robotic and/or laparoscopic surgery system, the retractor having a distal end and a proximal end, the proximal end located closer to a portion of the system located closer to a surgeon console, retractor effective to support in a generally uniformly distributed manner weight and/or massing of retracted tissue located within an animal, comprising:
an arm extendable from the system terminating in a fan mechanism having a plurality of overlapping slats for distributing weight of retracted tissue, so as to avoid injury to the retracted tissue during deployment of the retractor.

17. The retractor of claim 15, absent sharp edges.

18. A surgical procedure performed using the retractor of claim 1.

19. The procedure of claim 18, using a laparoscopic technique.

20. The procedure of claim 18, using a robotic surgical system.
